(19) United States
(12) Patent Application Publication
(10) Pub. No.: US 2022/0079509 A1
(43) Pub. Date: Mar. 17, 2022

(54) VACUUM DRESSING WITH CONTROL FEEDBACK

(71) Applicant: CARDIAC ASSIST HOLDINGS, Ann Arbor, MI (US)

(72) Inventors: Barry N. Gellman, Ann Arbor, MI (US); Kurt A. Dasse, Ann Arbor, MI (US); Allen Kantrowitz, Ann Arbor, MI (US)

(73) Assignee: CARDIAC ASSIST HOLDINGS, Ann Arbor, MI (US)

(21) Appl. No.: 17/473,134

(22) Filed: Sep. 13, 2021

Related U.S. Application Data
(60) Provisional application No. 63/077,190, filed on Sep. 11, 2020.

(51) Int. Cl. A61B 5/00; A61F 13/02

(52) U.S. Cl. CPC A61B 5/445; A61B 5/4836; A61F 13/0216; A61B 2560/0242; A61B 2562/0247; A61B 2562/0271; A61B 2562/029

(57) ABSTRACT

A wound management system (WMS) is provided that includes dressings, bandages, or implantable medical devices that are equipped with filters, environmental controls, and sensors that promote the formation of a natural biologic seal between the skin and the dressing to form a barrier to microbial invasion into the body that accelerates healing and mitigates wound or exit site infection. Percutaneous access devices (PAD) used with the WMS or other devices including peritoneal dialysis (PD) catheters, Steinman pin, Kirschner wires, and chronic indwelling venous access catheters that require skin penetration. The WMS minimizes risk of exit site infection by reducing the bioburden in the exit tunnel environment in the acute and subacute phases of the PD catheter post-implant. Visualization of the wound without taking off the dressing is provided via a window in the wound area or exit-site to visually monitor for signs of infection and the presence of exudate.

### CROSS-REFERENCE TO RELATED APPLICATION

This application claims priority of U.S. Provisional Patent Application Ser. No. 63/077,190 filed Sep. 11, 2020, which is incorporated herein by reference.

### FIELD OF THE INVENTION

The present invention in general relates to medical devices and systems for wound care and healing of chronic wounds, such as bedsores, venous stasis ulcers, burns, vascular insufficiency wounds, or surgically created entry sites for percutaneous access devices (PAD), other implantable medical devices, or surgical incisions and in particular to medical devices and systems for wound care and healing having atmospheric control feedback to encourage and maximize the rate of healing and skin formation in a wound area.

### BACKGROUND OF THE INVENTION

The body's natural wound healing process can be described as a complex series of events beginning at the moment of injury. Initially, in the hemostasis phase, the body reacts by delivering serous exudate, proteins and other factors to the wound to minimize the damage. Blood vessels, disrupted at the site of injury, seal by vasoconstriction and local clot formation to limit blood loss. In the next “inflammatory phase, immune system cells engulf bacteria and, exogenous foreign materials and endogenous local tissue debris to be carried away from the wound site. Next, the body begins to repair itself in a stage of healing often referred to as the proliferate proliferative phase. The proliferate phase, characterized by the delivery of wound fibroblasts, extracellular collagen and neovascular tissue needed to satisfy the metabolic demands of the healing wound, creates “of granulation tissue” in the wound bed. Granulation tissue provides a base structure over which additional cells may migrate to fill the wound tissue defect. Finally, the process matures during a “remodeling phase” as collagen gives wound fibroblasts contractile elements are activated and extracellular collagen polymers are secondarily modified, giving mechanical stabilization and strength to the scar tissue. Ultimately, the neovascularization recedes.

One technique for promoting the natural healing process, particularly, but not exclusively during the proliferate phase, is known as negative pressure wound therapy (VWTNPWT). Application of a reduced pressure, e.g., sub-atmospheric, to a localized reservoir over a wound has been found to assist in closing the wound. The reduced pressure may be effective to promote blood flow to the area, to stimulate the formation of granulation tissue and the migration of healthy tissue over the wound by the natural process. Also, a reduced pressure may assist in removing fluids exuding from the wound, which may inhibit bacterial growth. This technique has proven effective for chronic or non-healing wounds, but has also been used in for other purposes such as post-operative wound care and wound care of entry sites of implantable medical devices. As for example detailed in Huang C, Leavitt T, Bayer L R, Orgill D P. Effect of negative pressure wound therapy on wound healing. Current Problems in Surgery. 2014 July; 51(7):301-331.

Existing NPWT protocol provides for the introduction of a filler material into the wound to absorb exudates and promote fluid transport away from the wound bed. The wound filler may include such materials as non-reticulated foams, non-woven reinforcements or gauze. The wound and the absorbent wound filler material may then be covered by a flexible cover layer having an adhesive periphery that forms a substantially fluid tight seal with the healthy skin surrounding the wound. The cover layer thus defines a vacuum reservoir over the wound where a reduced pressure may be maintained over time by individual, periodic, or cyclic evacuation procedures.

An aspect of concern in existing VWTNPWT treatment is the management of forces generated in the dressing when a reduced pressure is applied. These forces may undesirably have effects such as deforming a flexible cover layer, drawing the peri-wound margins into the wound, putting the surrounding skin in tension, or inhibiting the wound healing cascade. These same forces may significantly compress the absorbent filler such that it forms a rigid mass. In such a state, the filler adopts an increased tendency to adhere to the wound bed, restricts the fluid passages available for exudate transport and inhibits penetration of the reduced pressure there through. An additional aspect of concern in existing NPWT treatment is the burden placed on nursing staff associated with monitoring the wound, vacuum reservoir, the absorbent wound filler and changing components and dressings to ensure proper healing of the wound. Not only do such monitoring and care requirements create a time and resources burden for nursing staff, but such necessary requirements are also counterproductive to wound healing in that in each instance of removing the bandage or vacuum reservoir subjects the wound to mechanical forces that can re-open partially healed wound margins of the would and expose the wound to an uncontrolled environment and potential for infection.

Entry sites for percutaneous access devices (PAD), vacuum dressings, or other implantable medical devices are susceptible to bacterial growth, infection, and problems healing, as described above with regards to chronic and non-healing wounds. Intravenous catheters act as an ingress situs sites for microorganisms, leading to biofilm formation and infection at the site of insertion or along the surface of the device. Infection of the catheter hub and catheter-related bloodstream infections are major complications for patients with indwelling catheters (e.g., Safdar and Maki, Intensive Care Med. 2004 January; 30(1):62-7; Saint et al., Infect Control Hosp Epidemiol. 2000 June; 21(6):375-80). The aforementioned infections are commonly referred to a skin exit site infection (SESI) and are common complication associated with long-term medical treatment modalities that require a catheter to penetrate the skin, including peritoneal dialysis, long-term vascular access, and drivelines associated with mechanical cardiac assistance as shown in FIGS. 1A and 1B. SESI have been associated with (1) Cellulitis of subcutaneous tissues; (2) erosion of tissues adjacent to skin exit site; infection along the catheter to deeper planes; and (3) intractable infection and systemic sepsis. SESI is a leading cause of unplanned hospitalizations for peritoneal dialysis (PD) patients.

Wound healing may be conceptualized to occur in four overlapping phases: (1) hemostasis; (2) inflammatory; (3) proliferative; and (4) maturation/remodeling. The long-term presence of the PD catheter at the skin exit site alters the course of these wound healing events resulting in the formation of a chronic wound, herein referred to as the “subcutaneous tunnel gap” between the wall of the PD catheter and the adjacent tissues in the vicinity of the PD catheter such as the subcutaneous fat, dermal and epidermal tissues. The unique wound conditions present in the tunnel gap define a microenvironment that predisposes the patient to recurrent SESI. Critical considerations for effective clinical management of the PD catheter exit site wounds and the tunnel gap include: reducing sources of reactive wound exudate and bacteria (bioburden) within the tunnel gap and minimizing repetitive mechanical injury; promoting granulation tissue infiltration of the PD catheter with a DACRON® cuff that with maturation of these granulations, collagen fibers anchor the DACRON® cuff to the neighboring subcutaneous tissues and stabilize the DACRON® cuff against longitudinal, rotational, and off axis trauma modes; and minimizing the risk of subcutaneous cellulitis (a condition shown in FIG. 2); and as shown in FIG. 3 supporting marsupialization of the walls of the exit tunnel resulting from epidermal migration down to the skin exit site (Phase 1), along the subcutaneous tunnel (Phase 2), down to the DACRON® cuff (Phase 3), and the deeper surface of the PD catheter (Phase 4). When a wound fails to complete the normal sequence of healing events, a chronic open wound without anatomical or functional integrity results.

Prior attempts at controlling catheter-related infection were directed to sterilization techniques such as by topical or fluidic antibacterials applied to the insertion site or integrated into the catheter itself. The antimicrobial lytic activity of C₁-C₈ alcohols is well known. Isopropyl alcohol at a concentration of 60-70% is widely used as an antimicrobial agent for sanitization of surfaces and skin. A concentration of 10% ethyl alcohol inhibits the growth of most microorganisms, while concentrations of 40% and higher are generally considered bactericidal (Sissons et al., Archives of Oral Biology, Vol. 41, 1, JN 1996; 27-34). Alternatively, commercial securement dressings such as the 3M 1657 dressings position a CHG-infused hydrogel pad at the skin exit site in an effort to draw away inflammatory fluid into the hydrogel pad and provide topical dosing of CHG as an antiseptic. Ong C T, Zhang Y, Lim R, et al. Preclinical Evaluation of Tegaderm™ Supported Nanofibrous Wound Matrix Dressing on Porcine Wound Healing Model. Adv Wound Care (New Rochelle). 2015; 4(2):110-118.

Catheters and other in-dwelling medical devices can be kept in place for as little as a few seconds for drainage or delivery of a substance. Besides catheters, other such devices illustratively include cannulas, lines for left ventricular assist devices (LVADs) chest tubes, and the like. It is increasingly common, however, for such devices and specifically peripherally inserted central catheters (PICC), skeletal guide wires, cardiac assist device lines, to be kept in place for weeks, months, or even years. The increased time in which such devices are maintained across the skin increases the likelihood of tunnel gap related infection adjacent to such devices.

Another common implantable device that breaks the skin and may be a source of infection are blood pumps that may be surgically implanted in, or adjacent to the cardiovascular system to augment the pumping action of the heart. The blood pump is sometimes referred to as a mechanical auxiliary ventricle assist device, dynamic aortic patch, balloon pump, mechanical circulatory assist device, or a total mechanical heart. Alternatively, the blood pump can be inserted endovascularly. Typically, the blood pump systems include a driveline that serves as a power and/or signal conduit between the blood pump internal to the patient and a controller/console external to the patient. Additional external medical devices may illustratively include implantable pumps such as insulin pumps and ECMOs (Extra-corporeal membrane oxygenator).

Percutaneous access devices (PAD) have been introduced that serve as semi-permanent or extended entry points for the aforementioned catheters and implantable and externally worn medical devices. For example, a percutaneous access device (PAD) may be surgically implanted in the body at the location in the skin where the driveline penetrates the skin to provide a through-the-skin coupling for connecting the supply tube to an extra-corporeal fluid pressure source. In a further example, electrical leads from electrodes implanted in the myocardium are likewise brought out through the skin by means of the PAD. Percutaneous access devices may also illustratively be used for other devices including peritoneal dialysis catheters, Steinman pin, Kirschner wires, and chronic indwelling venous access catheters that require skin penetration. More generally, medical appliances which are implanted so as to cross the skin surface and therefore violate the “barrier function” of the skin, may also illustratively be used for other medical purposes including peritoneal dialysis catheters and, chronic indwelling venous access catheters, neurologic prostheses, osseointegrated prostheses, drug pumps, and other treatments that require skin penetration.

The use of percutaneous access devices inhibits penetration or complications due to the presence of an agent in a subject. As used herein an “agent” is illustratively: an infectious agent such as bacteria, virus, fungus, other organism; or foreign material. Illustrative examples of foreign material include: bandage; soil; water, saliva, urine, or other fluid; feces; chemicals; or other matter known in the art. Illustrative examples of infectious agents that are prevented from penetrating or produce complications include P. aeruginosa, E. cloacae; E. faecalis; C. albicans; K. pneumonia; E. coli; S. aureus; or other infectious agents. As used herein, the term “subject” refers to a human or non-human animal, optionally a mammal including a human, non-primate such as cows, pigs, horses, goats, sheep, cats, dogs, avian species and rodents; and a non-human primate such as monkeys, chimpanzees, and apes; and a human, also denoted specifically as a “human subject”.

With NPWT, a force is typically applied to a PAD to counteract fluid collection or flow along the subcutaneous tunnel gap. It is common for fluid to develop within the subcutaneous tunnel gap often beginning immediately after insertion. The presence of this fluid allows migration, flow, or other penetration of agents normally excluded by the intact skin to areas below the skin. The penetration by these agents may lead to development of infectious disease, inflammation at the site of insertion, or other unwanted complications. A force that is applied is illustratively a vacuum via a NPWT dressing. A vacuum illustratively prevents fluid from accumulating with the subcutaneous tunnel gap. The negative pressure of the vacuum allows the natural pressures of biological material or other atmospheric pressure to move unwanted material away from the areas adjacent to the site of insertion.

The surface of the driveline, or of the PAD used in cardiac assist system may have characteristics which promote the formation of a natural biologic seal between the skin and the device to form a barrier to microbial invasion into the body at the skin penetration site. However, a common problem associated with implantation of a percutaneous access device (PAD) is disrupted skin regeneration about the periphery of the device leading to the formation of a dysfunctional immunoprotective seal against infection. New cell growth and maintenance is typically frustrated by the considerable mechanical forces exerted on the interfacial layer of cells. In order to facilitate skin regeneration about the exterior of a PAD, subject cells are often harvested and grown in culture onto PAD surfaces for several days prior to implantation in order to allow an interfacial cell layer to colonize PAD surfaces in advance of implantation. Unfortunately, cell culturing has met with limited practical acceptance owing to the need for a cell harvesting surgical procedure preceding the implantation procedure. Additionally, maintaining tissue culture sterility and integrity is also a complex and time-consuming task.

As an alternative to cell culturing on a percutaneous access device, vacuum assisted wound treatment about a percutaneous access device has been attempted. While DACRON® based random felt meshes have been used a fibroblast-adhesion feature promoting fibroblast attachment between the surface of the medical device and the adjacent tissues—such random felts have uncontrolled pore sizes that create safe havens for bacterial growth pockets beyond the reach of local cellular immune defense cells.

U.S. Pat. No. 7,704,225 to Kantrowitz solves many of these aforementioned problems by providing cell channeling contours, porous biodegradable polymers and the application of vacuum to promote cellular growth towards the surface of the neck of a PAD. The facilitating of rapid cellular colonization of a PAD neck allows the subject to act as their own cell culture facility, and as such affords more rapid stabilization of the PAD, and lower incidence of separation and infection.

The aforementioned PAD are constructed with one or more sleeves. A sleeve is optionally an inner sleeve or an outer sleeve. As used herein, the terms “inner” and “outer” are relative terms in terms of encompassing relative dimensions and should not be construed contextually as to positioning relative to the epidermis. An inner sleeve is optionally made of a porous material or scaffold that is optionally penetrated by fluids or gasses. A scaffold is optionally a tissue scaffold that allows or promotes attachment of cells, illustratively, fibroblasts to the surface of an inner sleeve. An inner sleeve is optionally treated. An inner sleeve treatment illustratively includes compounds or surface textures that promote attachment of fibroblasts or other cellular material. Optionally, the inner sleeve is made of a woven material. A woven material is optionally penetratable by cells, fluids, gas, or other materials.

It is appreciated that an inner sleeve is optionally the only sleeve present in a PAD. An inner sleeve is optionally a porous scaffold that is suitable for moving fluid or gas through the sleeve away from the surrounding environment. Materials operable for use as an inner sleeve illustratively include: collagen, PEBAX, nylons, polypropylenes, polyurethanes, polyethylenes (HDPE, UHWPE, LDPE, or any blend of the aforementioned polyethylenes), PET, NiTi, MYLAR, Nickel Titanium Alloy, titanium with manufactured micropores and microchannels, and other polymers such as other thermoplastic polymers, fabrics, silicones such as silicone rubber, latex, glass, or other materials known in the art. It is appreciated that polymeric materials with a gradient of cross-linking density through the material afford certain advantages with respect to promoting vacuum or hydrodynamic draw and fibroblast infiltration. By way of example, a polymer having a greater rigidity proximal to the central axis of the device relative to the distal surface inhibits pressure differential induced collapse. In some embodiments, an inner sleeve is made from chemically inert material. In some embodiments, the porous scaffold is in direct contact with the skin of the subject or traverses the skin of the subject. In some embodiments an inner sleeve is textured or woven in such a way so as to provide attachment sites for fibroblasts. A texture is optionally a nanotexture. Illustrative nanotextures have pore sizes that are uniformly less than 500 nanometers to provide an anchor point for a fibroblast pseudopod extension, while having dimensions that disfavor bacterial colonization. A nanotextured surface as used herein has features indentations of from 50 to 500 nanometer median dimension. In some embodiments, the indentations have a median dimension of between 100 and 300 nanometers.

In some embodiments of PAD, a texture is in the form of a scaffold. A scaffold is illustratively formed of gold. A gold scaffold is optionally formed by making a sleeve from a gold/silver alloy that is dipped in an acid such as a mineral acid which selectively dissolves the silver leaving a gold structure with appropriate porosity. Alternatively, a scaffold is formed from an acid etchable, biocompatible nanocrystal such as silver or silica is dispersed in a polymer melt such as polycarbonate and a neck either formed directly therefrom, or the nanocrystal-doped polymer is coated onto a neck substrate. Through subjecting the nanocrystal-doped polymer to an acid or base solution, depending on the solubility of the nanocrystal, voids are formed in the polymer reflective of the original nanocrystal dopant. For instance, silver is readily dissolved in 6 N hydrochloric acid while silica is dissolved in concentrated hydrofluoric acid. Dissolution in the presence of sonication is appreciated to facilitate the process. Nanocrystal loading of 1 to 10 percent by weight, depending on the specific nanocrystal dimensions, is sufficient to achieve the desired uniformity and density of pores. A titanium mesh scaffold can be manufactured with controlled pore sizes and channel dimensions using 3D additive manufacturing techniques known to practitioners of the art. Other porous surfaces and methods of manufacture are illustrated in U.S. Pat. No. 7,704,225 and references cited therein, each of which are incorporated herein by reference in their entirety.

It is appreciated that an inner sleeve is optionally coated or impregnated with a first compound. Coating or impregnating optionally provides lubrication so as to ease insertion of the instrument into the skin. A compound optionally: is antibacterial such as those described in WO 2008/060380, the contents of which are incorporated herein by reference; resist or promote cellular adhesion; are anticoagulants or procoagulants; or other desirable compound.

A compound optionally includes factors operable to selectively promote fibroblast growth and/or decrease attachment of bacteria or other contaminants. A compound optionally promotes growth of cells such as fibroblasts. A coating optionally includes the compound fibroblast growth factor (FBF). Optionally, FBF is used in a coating along with insulin and/or dexamethasone. The presence of dexamethasone and/or insulin will promote multiple layer growth of fibroblasts on the surface of or within the pores of a sleeve.

Coating substances illustratively include cell growth scaffolding matrices as detailed in U.S. Pat. Nos. 5,874,500; 6,056,970; and 6,656,496; and Norman et al. Tissue Eng. 3/2005, 11 (3-4) pp. 375-386, each of which is incorporated herein by reference. An exemplary coating is a tissue scaffolding, poly-p xylylene, parylene and chemical modified versions of such coatings to enhance post-insertion stabilization. Chemical modifications illustratively include bonding of fibronectin and other molecules implicated in the healing process. While tissue scaffolding and polymers are readily applied by painting, dip coating and spraying, it is also appreciated that polymeric coating are also readily applied by gas phase deposition techniques such as chemical vapor deposition (CVD). A coating is optionally porous in order to enhance capillary draw. In some embodiments a coating is biodegradable. A coating optionally has pores typically of an average size of between 10 and 500 microns, optionally, of an average size of between 30 and 50 microns.

An outer sleeve of a PAD functions to segregate or deliver vacuum draw pressure to an inner sleeve. The outer sleeve optionally circumferentially and longitudinally covers an inner sleeve. This configuration optionally shields the inner sleeve from epidermal bacterial or other agents upon insertion. An outer sleeve is optionally tapered at one or both ends. Tapering at a distal end (the end nearest the internal end of the catheter during use) provides improved insertion of the instrument into the skin of a subject. A taper may form a smooth interaction with the catheter at the outer sleeve distal end or a ridge is optionally present at or near the site of device interaction with the catheter. An outer sleeve is optionally made of any material suitable for use with a percutaneous instrument. Illustrative materials operable for an outer sleeve include such materials that have a memory or are self-expanding. Materials operable for use as an outer sleeve illustratively include: PEBAX, nylons, polyurethanes, polyethylenes (HDPE, UHWPE, LDPE, or any blend of the aforementioned polyethylenes), PET, NiTi, MYLAR, Nickel Titanium Alloy, other polymers such as other thermoplastic polymers, fabrics, silicones such as silicone rubber, latex, glass, or other materials known in the art. An outer sleeve optionally includes or is formed of a scaffold. An outer sleeve scaffold is optionally made of the same or different material as an inner sleeve scaffold. Scaffolds operable for an inner sleeve are similarly operable for an outer sleeve.

It is appreciated that an outer sleeve of the PAD is optionally coated or impregnated with a second compound. A second compound is optionally the same as a first compound. Coating or impregnation optionally provides lubrication so as to ease insertion of the instrument into the skin. A compound optionally: is an antibacterial coating or impregnated material such as those described in WO 2008/060380, the contents of which are incorporated herein by reference compounds to resist or promote cellular adhesion; anticoagulants or procoagulants; or other desirable compound.

In some embodiments of the PAD, an outer sleeve is textured. A texture is optionally formed of a tissue scaffold. A texture on an outer or inner sleeve optionally has pore sizes, ridges, depressions, indentations, or other texture that is uniform or non-uniform. A texture is optionally of a depth less than 500 nanometers to provide an anchor point for a fibroblast pseudopod extension, while having dimensions that disfavor bacterial colonization. A nanotextured surface as used herein has a uniform distribution of 50 to 500 nanometer median dimension indentations. In some embodiments, the indentations have a median dimension of between 100 and 300 nanometers.

In some embodiments of the PAD an outer sleeve surrounds an inner sleeve. The outer sleeve and inner sleeve are optionally formed from a unitary piece of material. The outer sleeve is optionally oriented surrounding an inner sleeve and optionally is slidably positionable about an inner sleeve. In some embodiments an outer sleeve protects an inner sleeve upon insertion of the inventive instrument and is positionally adjusted relative to the inner sleeve illustratively to a mark or other region that is optionally positioned above the epidermis. In some embodiments the inner sleeve remains traversing the skin while the outer sleeve is positioned above the epidermis or penetrates to one or more desired depths or levels.

An outer sleeve of PAD is optionally positioned external to the skin or near the surface of the skin when the device is employed. It is appreciated that an outer sleeve optionally forms an upper chamber that provides uniform distribution of vacuum pressure into and throughout the inner sleeve or the upper surface thereof. An outer sleeve optionally terminates in or is integral with a collar. A collar is optionally in fluidic connection with a conduit. In some embodiments a collar is made of a material with increased rigidity relative to an outer sleeve.

Without intending to be bound to a particular theory, a surface of a PAD in contact with compromised skin for device insertion promotes intercalation of fibroblasts regardless of whether the surface is textured, coated, or a combination thereof so as to simultaneously promote orthological changes in the fibroblast from circulatory form to dendritic and/or stellate forms through a depth of more than one layer of fibroblast at a time and preferably more than five layers of fibroblasts simultaneously anchoring to the device and more preferably more than ten such layers of fibroblasts. Fibroblast orthological changes simultaneously in more than one layer of such cells serve to rapidly stabilize the percutaneous inventive device. In conjunction with the vacuum pressure draw during the process, infection risks are minimized and a PAD is stabilized against pullout or other device motions relative to the surrounding dermal layers.

A PAD optionally includes one or more gaskets or seals. A seal prevents vacuum pressure from escaping to the atmosphere or from drawing bodily fluid into the system from the subcutaneal end of the instrument. A gasket is optionally made from any material suitable for creating a seal around the circumference of a catheter. A gasket is illustratively made from silicon rubber, latex, nylon, or other polymeric materials. A gasket is optionally connected to or integral with an outer sleeve, an inner sleeve, a bandage, or a collar.

A conduit is optionally fluidly connected to an inner sleeve either via a gasket or direct connection. A conduit is optionally made of any material that will resist total collapse under vacuum pressures used with the invention. A conduit is optionally transected by a valve. A valve is operable to engage, disengage, or adjust the vacuum pressure translated to the inner sleeve. A valve is optionally mechanically or electrically controlled. Any valve or valve system known in the art is operable herein. A valve is optionally positioned at the junction between the conduit and the instrument portions of the PAD.

As mentioned a PAD may be connected to a vacuum source. A vacuum source can be any source operable for creating negative pressure in or around the device. A vacuum source is optionally a passive vacuum such as a vacuum tube or bottle, or an active vacuum source illustratively a mechanical pump, a syringe, or other vacuum source. A vacuum source optionally applies a continuous or intermittent negative pressure. The magnitude of the negative pressure is optionally adjustable, constant, or variable. In some embodiments an intermittent vacuum is used. Alternatively, a hydrodynamic draw agent is provided that draws fluid from the tissue surrounding through the sleeve via the conduit. A hydrodynamic draw source illustratively includes a super absorbent polymer such as sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile; high osmotic pressure compositions, such as water soluble salts; and capillary flow draw agents such as dry silica, or other dry hydrophilic powders such cellulosic material. It is noted that recently a diaper filler in the form of a polyacrylic acid as a superabsorbent polymer and a bactericidal skin wash chlorhexidine has been used to draw fluid from the tissue surrounding the sleeve via the conduit. However this approach to fluid draw has limited and exponentially decaying moisture draw.

FIG. 4 illustrates wearable and implanted components of an exemplary prior art cardiac assist system. A PAD 10 serves as an attachment point for an external supply line 12 that supplies air or fluid from a wearable external drive unit (EDU) 14. The EDU 14 is powered by a wearable battery pack 16. Inside the body of the patient, a drive line 18 is attached to the PAD 10 and provides an air or fluid conduit to a cardiac assist device 20.

FIG. 5 depicts a PAD generally at 100 as shown in U.S. application Ser. No. 13/416,546 to Kantrowitz. A cap 102 is formed of a material such as silicone, a polymer or a metal and serves to keep debris from entering the device 100. Preferably, the cap 102 is remote from the surface of the epidermis E. The medical appliance 34 depicted as a catheter and vacuum or hydrodynamic draw tubing 104 pass through complementary openings 106 and 108, respectively formed in the cap 102. The tubing 104 provides fluid communication between a vacuum or hydrodynamic draw source 22 and an inner sleeve 12d. The inner sleeve 12d is characterized by a large and rigid pore matrix 18 in fluid communication to a vacuum source 22 such that the source 22 draws (arrow 22D) tissue fluid and fibroblasts 21 into the sleeve 12d. Sleeve 12d has a surface 24 that is optionally nanotextured to promote fibroblast adhesion. The surface 24 is optionally decorated with a pattern of contoured cell-conveying channels. It is appreciated that inner sleeve 12d optionally includes matrix 26 thereover, a coating substance 27, or a combination thereof. The coating 27 is appreciated to need not cover the entire surface 24. The tissue contacting surface 29 of substance 27 is optionally nanotextured. A flange 112 is provided to stabilize the implanted device 100 within the subcuteanous layer S. A flange 112 is constructed from materials and formed by methods conventional to the art. For example, those detailed in U.S. Pat. Nos. 4,634,422; 4,668,222; 5,059,186; 5,120,313; 5,250,025; 5,814,058; 5,997,524; and 6,503,228.

FIGS. 6A-6C illustrate a modular external interface housing 200 coupled to the PAD 100 as disclosed in U.S. application Ser. No. 15/555,952 to Subilski. The modular external interface 200 forms a collar about the neck 110 of the PAD 100 with the main body 216 with a locking feature 218, such as a male extension that engages a female receptacle or cavity as a mechanical overlap connection. In a specific embodiment the main body 216 is made of silicone. The collar seal between the main body 216 and the neck 110 of the PAD 100 forms a hermetic seal with a gasket 230, which in a specific embodiment is a flexible gasket integrated into the main body 216. In a specific embodiment the gasket 230 may be a floating gasket. The stabilization of the PAD 100 within the skin to form a germ-free barrier requires subject cells to grow onto the neck surfaces 17 as shown in FIG. 5 of the PAD 100 adjacent to the subject's epidermis E. The neck surface region 17 is adapted to promote growth of autologous fibroblast cells thereon. A suitable exterior side surface substrate for fibroblast growth is a nanotextured polycarbonate (LEXAN®). The modular external interface 200 has a central opening adapted for at least one drive line 220 for insertion into a PAD, and a portal 224 for a vacuum line 222.

The modular external interface 200 is secured and sealed to an outer layer of a patient's skin with a medical dressing. In a specific embodiment the medical dressing is a preform patterned and shaped to conform to the exterior of the modular external interface 200. In a specific embodiment the medical dressing preform may be in two halves (212, 214) that overlap. In a specific embodiment the medical dressing preform may be transparent. In a specific embodiment the medical dressing preform may be made of Tegaderm™ manufactured by Minnesota Mining and Manufacturing Company.

U.S. patent application Ser. No. 15/125,273 entitled “Active Hermeticity Monitoring” to Kantrowitz et al. provides a system and method for measuring and monitoring wound hermaticity and correlating a hermaticity measurement with the establishment of intact biological barrier function of the stratum corneum layer of skin. Embodiments of the method and system for actively assessing hermaticity in wound closure are incorporated into the design of percutaneous skin access devices (PAD), bone anchors, or a wound dressing or bandage alone without at PAD. In a specific embodiment, the hermaticity of the skin wound in the vicinity of the skin-PAD interface is measured as a function of the fluid exudate or transudate egressing from the skin wound in the vicinity of the skin-PAD interface.

As disclosed in U.S. patent application Ser. No. 15/125,273 the degree of hermaticity is related to impedance measurements performed on the skin of a patient. In a specific embodiment, an active impedance measurement may be performed as described in “Impedance measurements of individual surface electrodes” (Medical and Biological Engineering and Computing, November 1983, S. Grimnes) with two electrodes around the collar of a PAD, and/or the active impedance measurements incorporated into the PAD device. Impedance measurement of resistance (R), reactance (Xc), and phase angle (PA) have been shown to be effective in monitoring wound healing closure and infection as disclosed in “Bioelectrical Impedance Assessment of Wound Healing” (Journal of Diabetes Science and Technology, January 2012, Lukaski et al.). As a wound heals resistance (R), reactance (Xc), and phase angle (PA) values increase, and if the wound is infected the values drop. Additional electrode patterns are possible which could further enhance the usefulness of the information.

Furthermore, an assessment of hermaticity may be determined with measurements of humidity in the vacuum line to a PAD. The humidity readings may be taken with impedance humidity sensors. In still other embodiments, local tissue oxygenation in the immediate vicinity of the PAD or other measurements may be used to determine wound healing.

The hermaticity sensor measurement information is readily employed for local closed-loop control of the vacuum supply to the PAD, and to alert the patient with regards to progress or problems with the PAD-skin interface. Additionally, the hermaticity information may be transmitted wirelessly to medical personnel to allow for remote monitoring of the healing wound. For example, as impedance or humidity in a vacuum line stabilizes, medical personnel may be notified that the wound has healed. Alternatively, if the impedance or humidity deviated from expected values, medical personnel could be notified that there may be an infection or a mechanical disruption to the wound; alarms could also be set to notify the patient. In an embodiment, the vacuum supplied to the PAD could automatically be increased or decreased based on the wound healing.

Despite the advances in PAD design and the securement of PAD to a subject's skin there continues to be a problem of disrupting the formation and maintaining of skin layers about the PAD with respect to flexible or pliable drivelines during the healing process. In addition, while vacuum pumps, capillary draw, and hydrodynamic draw have been used reduce the pressure on the insertion site and thereby dry the insertion site to stimulate granulation that will mechanically stabilize the appliance and reduce the prospect of infection; infection at the site of PAD used with pliable and flexible drivelines continues to occur as the seal between the layers of skin and the bendable driveline tends to either not fully form or fails as the driveline flexes at the insertion site. Furthermore, there continues to be a need to adjust pressure to preclude skin prolapse (pucker) around a PAD.

Second Tubing

Pressure Sensor

Thus, there is a continuing need for improved methods for healing chronic, non-healing, or implant entry site wounds and devices that are equipped with improved environmental controls, pressure controls, and feedback that encourage and expedite nascent layers of skin that are being formed during the healing process, as well as maintaining an infection preventive seal around percutaneous access devices. There is a further need for an effective wound management system to mitigate infection and accelerate healing of such wounds to increase safety, improve quality of life, reduce healthcare costs, and lessen the burden of wound care on nursing staff and caregivers.

### SUMMARY OF THE INVENTION

A wound management system (WMS) is provided for monitoring and controlling environmental conditions of a wound area of a patient. The WMS includes a wound dressing configured to be positioned over the wound area, the would dressing defining a wound environment, and at least one of a humidity sensor, a pressure sensor, a temperature sensor, and a chemical sensor integrated into the wound dressing to provide physiologic parameters that correlate to a degree of wound healing. The WMS further includes a pump in fluid communication with the wound environment, and a controller configured to control operation of the pump.

A method is provided for measuring and monitoring wound conditions of a patient. The method includes placing one or more sensors for measuring parameters that correlate to a degree of wound healing at a wound area of a patient, where the one or more sensors are incorporated into a wound dressing positioned to cover the wound area. The one or more sensors determine the degree of wound healing via measurements of humidity, temperature, air quality, and pressure in a vacuum line to the wound dressing.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIGS. 1A and 1B are photos of driveline infection at a skin exit site in a heart failure patient supported by a prior art left ventricular assist device (LVAD);

FIG. 2 is a photo of an exit site infection illustrating the catheter, contaminated exit site, and subcutaneous location of subcutaneous cellulitis;

FIG. 3 illustrates the four phases for marsupialization of walls of a tunnel: Phase (P)1: beginning of downward epidermal migration; P2 along the subcutaneous tissue, P3 down to the cuff, and P4 the deeper portion of the catheter;

FIG. 4 illustrates prior art wearable and implanted components of a cardiac assist system with a percutaneous access device (PAD) and internal driveline;

FIG. 5 is a prior art, partial cutaway view of a flanged percutaneous access device (PAD) with relative dimensions of aspect exaggerated for visual clarity;

FIGS. 6A-6C are perspective views of a prior art modular external interface seal for a PAD appliance secured with adhesive dressings to a subject;

FIG. 7A illustrates a negative pressure wound dressing in a low vacuum state of approximately 30 mm Hg in accordance with an embodiment of the invention;

FIG. 7B illustrates a negative pressure wound dressing in a high vacuum state of approximately 125 mm Hg in accordance with an embodiment of the invention;

FIG. 8 illustrates an embodiment of the inventive wound management system (WMS);

FIG. 9 is an exploded view of the WMS of FIG. 8;

FIG. 10 illustrates an alternative dressing for the WMS of FIG. 8;

FIG. 11 illustrates a dressing humidity and temperature sensor along with a slow leak valve positioned within the mechanical structure of the WMS of FIG. 8; and

FIG. 12 is a schematic diagram illustrating an overall view of communication devices, computing devices, and mediums for implementing embodiments of the invention.

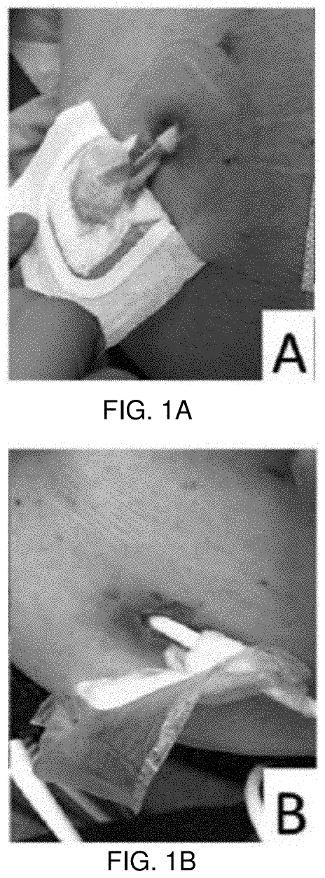

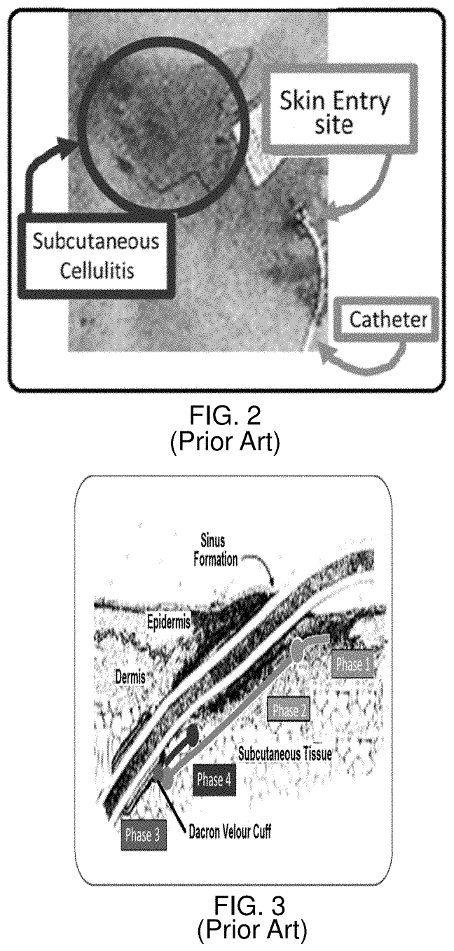

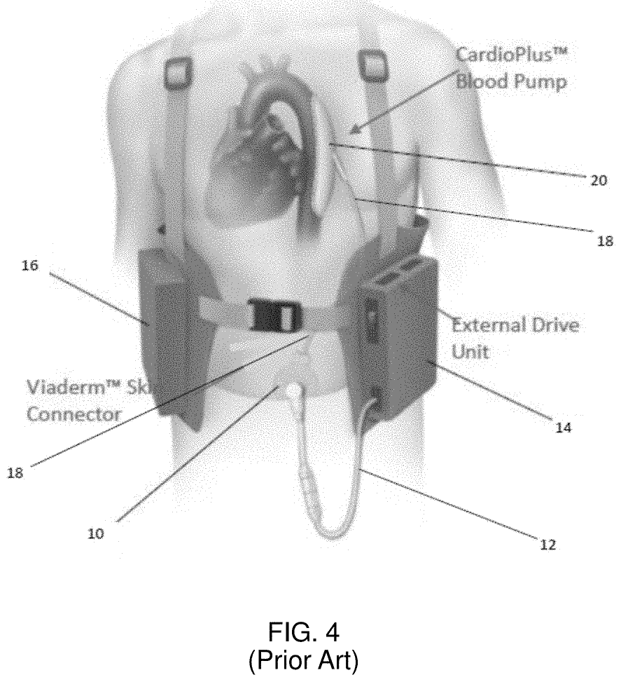

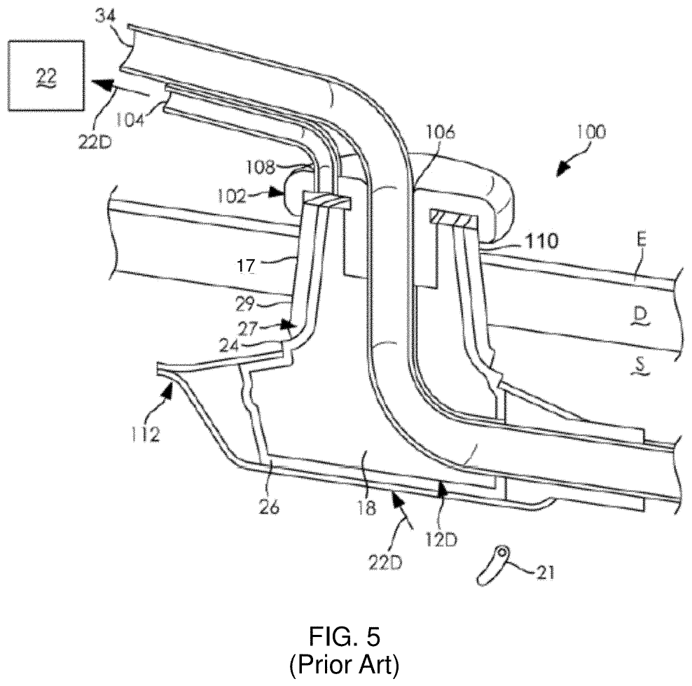

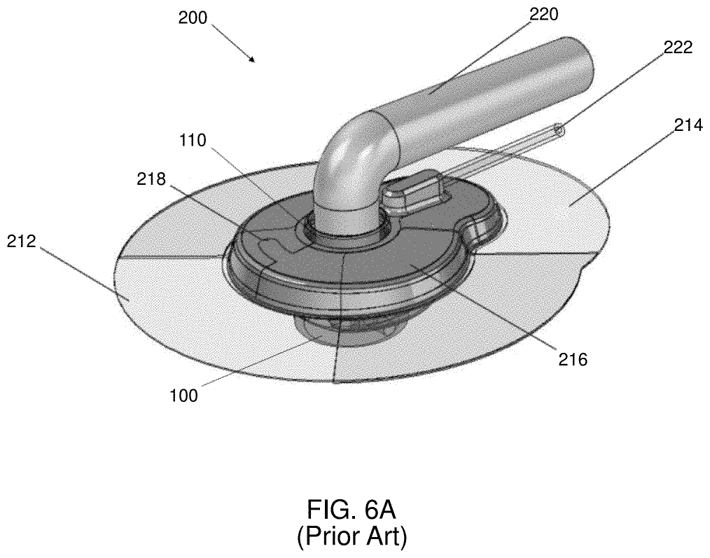

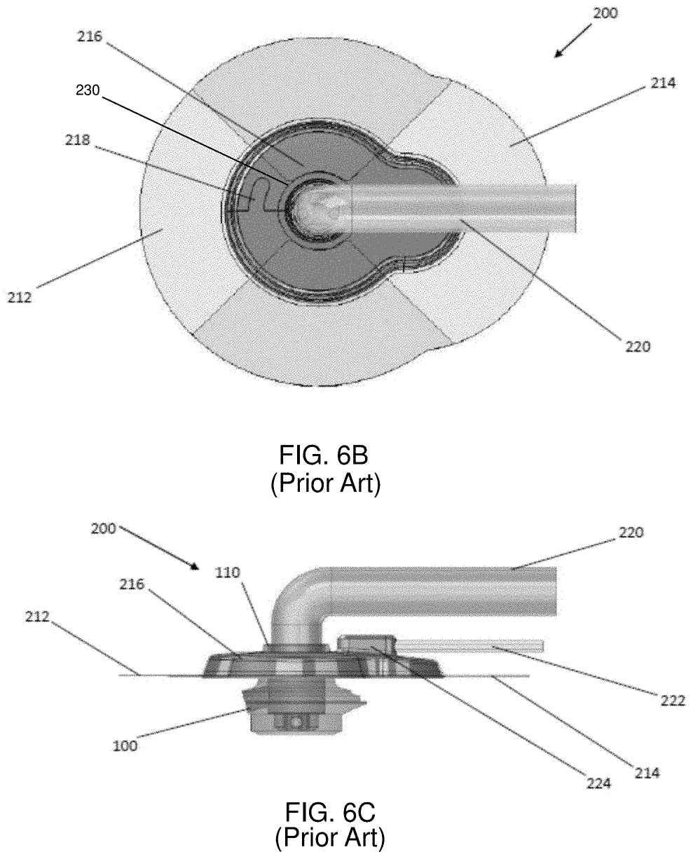

### DETAILED DESCRIPTION OF THE INVENTION

Embodiments of the invention provide a wound management system that includes dressings, bandages, or implantable medical devices that are equipped with filters, environmental controls, and sensors that promote the formation of a natural biologic seal between the skin and the dressing to form a barrier to microbial invasion into the body that accelerates healing and mitigates wound or exit site infection. Percutaneous access devices (PAD) may also illustratively be used with the inventive wound management system or other devices including peritoneal dialysis (PD) catheters, Steinman pin, Kirschner wires, and chronic indwelling venous access catheters that require skin penetration.

It is noted that previous efforts have concentrated on removing moisture or humidity from wound areas, however a level of moisture is required to allow fibroblasts to actively attach to an implanted PAD and to promote the establishment of intact biological barrier function of the stratum corneum layer of skin and for wound healing in general. It is also noted that moisture and pressure levels may be needed to change as the wound healing process progresses through different stages. It is further noted that pressure levels may require adjustment to preclude skin prolapse around an implanted device or improper wound healing.

Embodiments of the inventive wound management system (WMS) are designed to improve wound healing while minimizing the risk of wound area infection by enhanced wound monitoring using sensors, providing for visual inspection without a need to remove the dressing, and providing moisture and temperature controls. According to embodiments, the WMS minimizes risk of exit site infection by reducing the bioburden in the exit tunnel environment in the acute and subacute phases of the PD catheter post-implant. Currently available wound dressings do not permit visualization of the exit site, which risks delayed detection of infection until the dressing is removed, and such removal of existing wound dressings can be counterproductive to the proper and speedy healing of the wound in that removal of dressings applies undue forces to the wound and exposes the wound to potential infection from the environment. To address this clinical need, the inventive WMS enables visualization of the wound without taking off the dressing by providing the following features. A window to visualize the wound area or exit-site for signs of infection and the presence of exudate. These observations assist the caretakers in determining whether to change the dressing. According to embodiments, strain relief for a catheter is present to limit repetitive trauma due to movement, tension, and twisting of the catheter during the healing process. The ability to measure and monitor pressure, humidity, and temperature at the wound area or exit site, and closed-loop vacuum control feedback to maintain a constant humidity level (92% relative humidity). A controlled localized vacuum (−125 mm Hg) at the wound area or exit-site while avoiding exposing the neighboring normal epidermis to vacuum and the risk of maceration. Intermittent cycling of the vacuum to evacuate bioburden from the wound area or exit tunnel. An intentional controlled slow leak within the dressing to allow air exchange within embodiments of the inventive WMS. The ability to maintain a vacuum level set-point to keep the tissue surrounding the wound area or exit site in close approximation to the tissue/biomaterial interface to promote healing. A collection container (35 ml) to hold biohazard fluid to be discarded after use.

Embodiments of the inventive wound management system combine the use of vacuum assist technology with a novel wound dressing to control the environment within and around the wound area or medical device penetration/exit site. Embodiments of the inventive WMS employ algorithms to control humidity and temperature to apply appropriate vacuum levels. The humidity and temperature control algorithms, combined with a slow leak approach allow air exchange in the dressing, and represent a new innovative approach to wound care. Unlike other negative pressure wound dressings, embodiments of the inventive wound management system (WMS) are specifically designed to treat the local wound conditions unique to the skin exit site of a catheter crossing the skin. Such embodiments of the inventive device are designed to stabilize passage of a catheter through a dressing with a strain relieving component to protect the exit site from trauma while applying negative pressure to treat the wound in a highly controlled manner. Embodiments of the inventive WMS provide a fully vacuum sealed system via injection molded and/or insert molded components to create the desired hermetic seal.

Embodiments of the inventive WMS include five core components: (1) a vacuum assist technology (VAT) wound dressing; (2) humidity, pressure, temperature sensors, and/or chemosensors identifying volatile compounds (such as sulfur-containing compounds and/or other chemotransducer-detectable compounds emitted from the wound into the gaseous environment of the NPWT dressing, integrated into the wound dressing system to provide physiologic parameters for time-varying control of the milieu surrounding the catheter exit site; (3) an exudate reservoir; (4) a pump; and (5) a controller. Embodiments of the inventive WMS also include a strain relieving fixture.

Embodiments of the inventive WMS employ a more compliant dressing with a unique design to accommodate the variation in catheter diameters produced by multiple manufacturers.

In specific inventive embodiments, a single-use controller has a microprocessor that receives input from the wound dressing sensors and compares the signals to the controller sensors to adjust the vacuum levels and/or to turn the vacuum on and off. The system controls a diaphragm pump/motor assembly powered by four (4) AA batteries, a display screen, an interlock for canister engagement, battery pack, pressure sensor, and a slow-leak flow restrictor. Alarms/Alerts provide visual and audible alerts to the user. The foam within the wound dressing provides a tactile/visual indication of vacuum ON/OFF and cycling conditions. The controller may be a direct current (DC) powered pressure regulator that delivers negative pressure to the wound dressing ranging from −30 mmHg to −125 mmHg. The set point (threshold) for the optimal percent humidity is a critical consideration when developing the humidity/temperature algorithm for automated control of the inventive WMS. In a specific inventive embodiment, the rationale for selecting 92% humidity is based on the need to maintain a moist environment surrounding the wound area or exit site wound while keeping the environment less than 100% humid. The percent humidity detected by the humidity sensor can be adjusted to any threshold over the entire range from 0% to 100%. According to embodiments, the WMS includes a tube configured to introduce moisture into the dressing in the event, for example the humidity sensors indicate that the humidity within the dressing, i.e. the environment surrounding the wound area or exit site wound, falls below the set point (threshold) for the optimal percent humidity. Other triggers could include information from the chemosensors indicating that infection may be of increasing importance in the wound melieu. Such information could be used to signal medical care personnel or trigger the release of cleansing agents, antiseptic agents, or antibiotic agents. The present invention thereby functions to monitor a wound healing environment and adjust that environment in response to a condition that is outside of a preselected threshold or detection of a pathogen or metabolite thereof. By way of example, a chemical sensor detecting specific compounds, such as amines or thiols, concentrations, or certain ratios thereof are indicative of the growth an anaerobic pathogens. In still other embodiments, an alarm is triggered in such a condition, a therapeutic administered, or a combination thereof in response to a potentially dangerous condition, as detailed below.

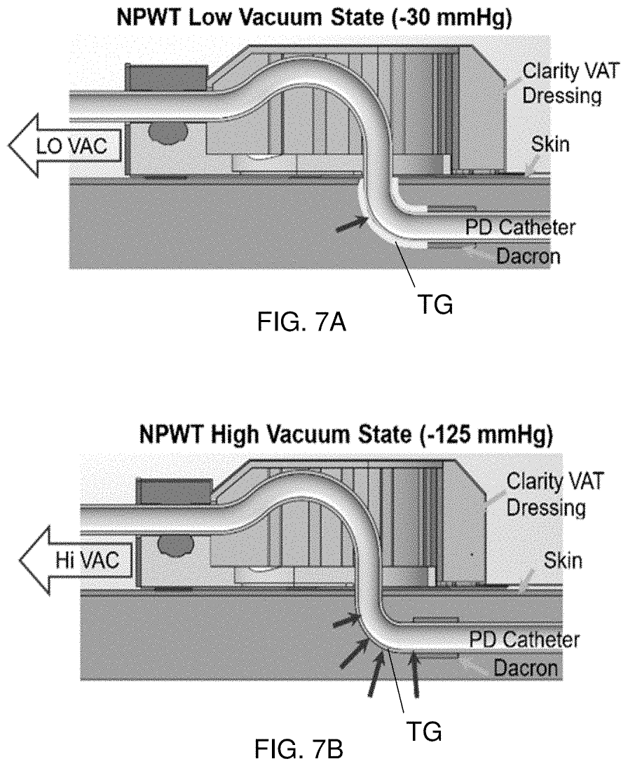

In embodiments of the inventive WMS, controlled negative pressure wound therapy (NPWT) is applied to aspirate wound exudate and bioburden carrying microorganisms out of the wound area or peri-catheter region while protecting the surrounding skin from harsh vacuum exposure. During a NPWT low vacuum state as shown in FIG. 7A, wound exudate accumulates within tunnel gap (TG). During a NPWT high vacuum state as shown in FIG. 7B, bioburden/exudate is expressed (as shown by multiple arrows) from within the diminished tunnel gap TG and the adjacent subcutaneous tissue is approximated to the contour of the DACRON® cuff and outer wall of PD catheter. In specific inventive embodiments the duration of use for each dressing will be up to seven days. Embodiments of the WMS are designed to promote rapid approximation of the tissue to the catheter by providing controlled vacuum up to approximately 125 mm Hg and both mechanical and vacuum-based stabilization of the composite construct of the PD catheter/adjacent tissues/WMS dressing during the healing process. The availability of an electronically modulable vent valve can allow both low frequency and high frequency modulation of the strength of the vacuum.

A central element of the inventive WMS is the vacuum assist technology (VAT) wound dressing that provides the benefits of NPWT to bear on wound management of the wound and in the vicinity of skin exit site of a PD catheter. The extensive investigations of the mechanism of action of NPWT by Orgill has led to the proposed four basic mechanisms of action: 1) macro-deformation or wound shrinkage; 2) microdeformation or micromechanical cellular changes at the wound-interface surface; 3) removal of fluids; and 4) maintenance of a moist wound environment. Orgill found that granulation tissue formation is affected by the time and frequency of application of vacuum to the wound environment.

Embodiments of the inventive WMS are designed to: (1) refresh the environment adjacent to the wound with air; (2) permit direct visualization of the wound to monitor healing and early signs of infection; (3) monitor relative humidity, pressure, temperature and/or the presence of chemotransducer-detectable biologically produced compounds emanating from within the external environment adjacent to the wound, (4) remove wound exudate/bioburden; (5) control and modulate vacuum relative to the pressure, humidity, and temperature with feedback control at the wound site; and (6) treat the wound area with anti-infection treatments when signs of infection have been detected.

Embodiments of the inventive WMS have integrated pressure, relative humidity, and temperature sensors to measure water vapor production adjacent to the wound area or skin entry site. Specific embodiments of the inventive WMS include integrated air sensors that monitor the gases within the wound environment for chemicals associated with infections. In such embodiments, the inventive WMS may additionally include an introducer configured to introduce anti-infection treatments, such as antibiotics, into the wound environment when the integrated air sensors detect chemicals associated with an infection in the air sampled from the wound environment. This allows the detected infection to be treated without removing the wound dressing. According to embodiments, a slow leak valve as shown in FIG. 11 enables air exchange within the dressing. A series of algorithms enable vacuum control of the wound environment based upon water vapor production metrics, as well as avoidance of unintended vacuum leaks throughout the dressing. Controllable flow restrictors are optimized for air exchange rates, and the algorithms compare humidity and temperature surrounding the wound to the conditions outside the wound to control the ON/OFF vacuum cycle to remove moisture and draw trapped fluid from around the wound site or any catheter that is present and the exit site and provide a fully vacuum sealed system. Visualization of the wound site is provided to a user through a window positioned above the wound area or access site.

Embodiments of the invention monitor and dynamically control levels of humidity and pressure to optimize wound closure about an implanted device or when a PAD is not present a wound itself at a wound dressing. Embodiments of the method and system for actively assessing wound closure are incorporated into the design of percutaneous skin access devices (PAD), bone anchors, or a wound dressing or bandage alone without at PAD. The pressure and humidity sensor provide active feedback for making changes to the ecology of the wound site or PAD insertion site. In specific inventive embodiments a filter, which illustratively includes a submicron filter, is used to aerate the wound while also preventing pathogens in the ambient air from reaching the wound.

In certain embodiments of the present invention, an assessment of hermaticity may be determined with measurements of humidity in the vacuum line to a wound dressing or PAD. The humidity readings may be taken with impedance humidity sensors. In still other embodiments, local tissue oxygenation in the immediate vicinity of the dressing or PAD or other measurements may be used to determine wound healing.

In certain embodiments of the present invention, an assessment of air quality may be determined with measurements of chemical sniffing sensors in the vacuum line to a wound dressing or PAD. These sensors are capable to sniffing the air in the vacuum line for chemicals associated with infection. The air quality readings may be taken with air sniffing sensors. Such an air sniffing/air quality sensor illustratively tests for oxygen, or sulfur; exudate biochemical such as electrolytes such as sodium, potassium, or chloride; small molecules such as urea, creatinine, fibrinogen, matrix metalloproteinases (MMPs); proteins such as tumor necrosis factor (TNFα) and C-reactive protein (CRP); and combinations thereof.

The hermaticity, temperature, and/or air quality measurement parameters are readily communicated by wired or wireless connection to a computing or communication device for immediate or remote monitoring. Known and future wireless standards and protocols such as, but not limited to, Bluetooth, Zigbee, WiFi, and others may be used to transmit hermaticity, temperature, and/or air quality measurements. Remote monitoring may be facilitated via an Internet or cellular network enabled device in communication with the output of a hermaticity measurement device or sensor. The hermaticity, temperature, and/or air quality measurement devices or sensors may require an external power source such as a battery, or may be passive elements such as radio frequency identification elements (RFID), which obviate the need for an electrical power source to be directly incorporated into the PAD or wound dressing. A passive RFID element retransmits a signal using the energy of an incoming interrogation signal, where in embodiments of the inventive hermaticity sensor, temperature sensor, and/or air quality sensor the transmitted signal will vary in frequency or phase with the respective measurement. In certain embodiments, battery power used to supply the vacuum source of the wound dressing or PAD may also be utilized to supply power to the one or more hermaticity, temperature, and/or air quality sensors.

The hermaticity, temperature, and/or air quality sensors measurement information is readily employed for local closed-loop control of the vacuum supply to the wound dressing or PAD, and to alert the patient with regards to progress or problems with the wound dressing or PAD-skin interface. Additionally, the hermaticity, temperature, and/or air quality information may be transmitted wirelessly to medical personnel to allow for remote monitoring of the healing wound. For example, as impedance or humidity in a vacuum line stabilizes, medical personnel may be notified that the wound has healed. Alternatively, if the impedance or humidity deviated from expected values or if an air quality sensors detects a chemical commonly associated with an infection, medical personnel could be notified that there may be an infection or a mechanical disruption to the wound; alarms could also be set to notify the patient. In an embodiment, the vacuum supplied to the wound dressing or PAD could automatically be increased or decreased based on the wound healing, moisture could automatically be introduced to the wound environment, and/or an anti-infection treatment could automatically be supplied to the wound environment.

According to embodiments, the dressing includes a fiber provided within the wound environment or interwoven with the dressing. The fiber illustratively includes monofilament, polyfilament, hollow fibers, or combinations thereof. A fiber modifies properties by affording capillary draw to promote drying, fibroblast infiltration, and in some circumstances monitoring of serous fluid for early indications of infection. Hollow fibers are particularly well suited for such sampling. It is appreciated that fibers, such as quartz fibers can be used to transmit biocidal ultraviolet light emissions, while hollow fibers can convey therapeutic fluids, or biocidal gases such as oxygen alone, or in combination with ozone.

In specific inventive embodiments, integrated multi-lumen tubing as disclosed in US Patent Publication US20200289810A1 to Kantrowitz is used for delivering a vacuum. Integrated multi-lumen tubing provides a combination of intravenous (IV) infusion lines, vacuum lines, and in some instances monitoring lines for attachment to a percutaneous access device or long term implant. The integration of the intravenous infusion lines, vacuum lines, and monitoring lines that connect to the wound dressing or PAD and other inserted instruments organizes the myriad of intravenous infusion lines, vacuum lines, and monitoring lines that connect to the wound dressing or PAD and other inserted instruments that tend to get tangled, interfere with patient comfort and movement, and are potentially difficult for health care workers to change and maintain. Furthermore, by using the lines associated with the IV already present in a hospital or medical facility allows for use of the existing vacuum source used in the facility.

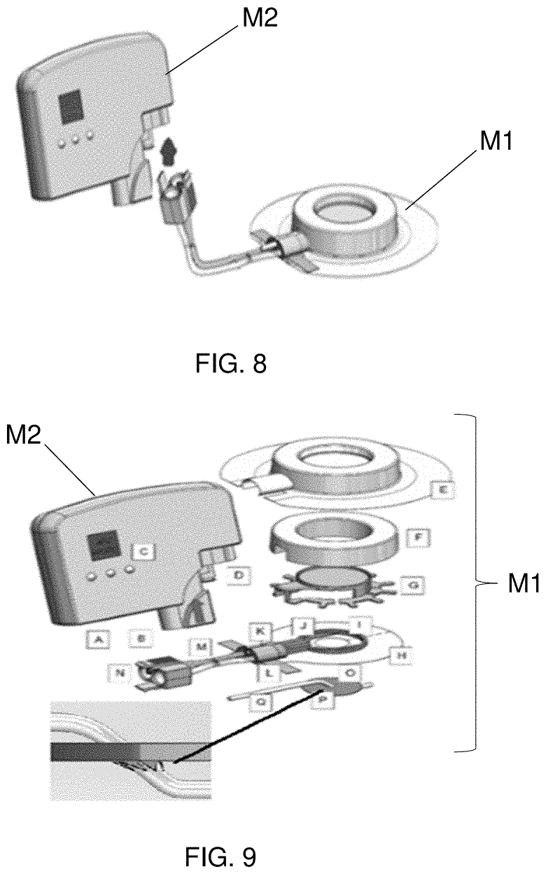

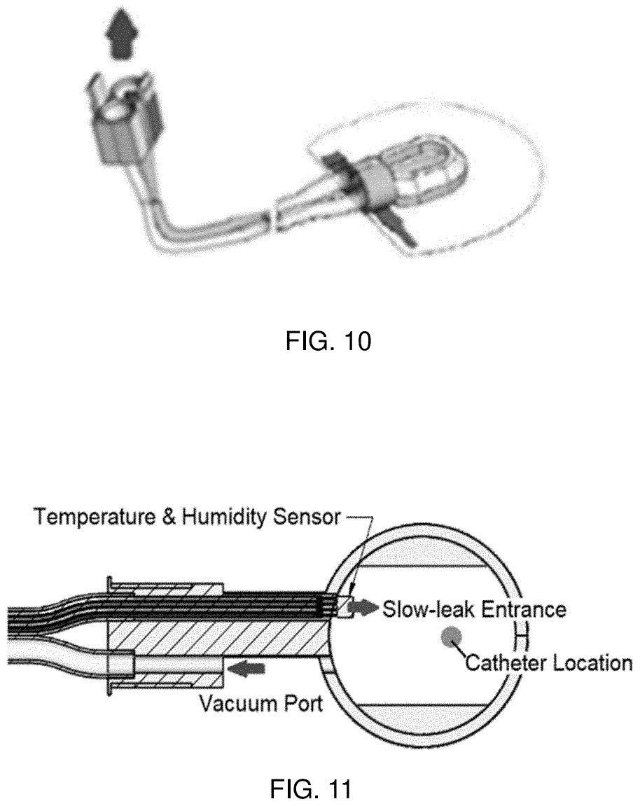

FIGS. 8 and 9 illustrate an embodiment of the inventive WMS. FIG. 10 illustrates an alternative dressing for the WMS of FIG. 8. The WMS has two primary modules. Module 1 (M1) is made up of the wound dressing that includes: (a) dressing that permits visualization of the wound and/areor exit-site through a window (G); (b) skin protection layers (H & O) that prevent necrosis of the surrounding skin; (c) restricting the application of vacuum only to the wound area or exit-site (O & P); (d) slow-leak port (D) that permits filtered air to be exchanged in the wound dressing to reduce bioburden; (e) humidity, air quality, and/or temperature sensors (J) to monitor relative humidity, air quality, and temperature and permit algorithm control of the wound environment via control of the temperature, introduction of moisture or anti-infection treatment via introduction port (I); (f) according to embodiments, integrated strain relief to secure a catheter, if used, during healing (K & L); and (g) dual supply line with a single connector to draw vacuum from the site and deliver replacement air (M & N). The inventive WMS skin dressing provides a means to enhance wound healing for patients with a chronic or non-healing wound or peritoneal dialysis catheter (Q).

Module 2 (M2) includes a battery powered controller with innovations that include: (a) reusable, quiet controller for 7-days of continuous use (A) that delivers controlled vacuum to the wound dressing; (b) integrated orifice flow restrictor and filter for wound air exchange (D & inside controller housing); (c) disposable exudate collection canister with filters to protect the controller from contamination (B); (d) optional system controls with a display to permit multiple modes of operation including intermittent, continuous and smart. The smart mode maintains optimum vacuum level based upon relative humidity and temperature sensors that permit cycling the vacuum and introduces moisture and/or anti-infection treatments to the wound environment.

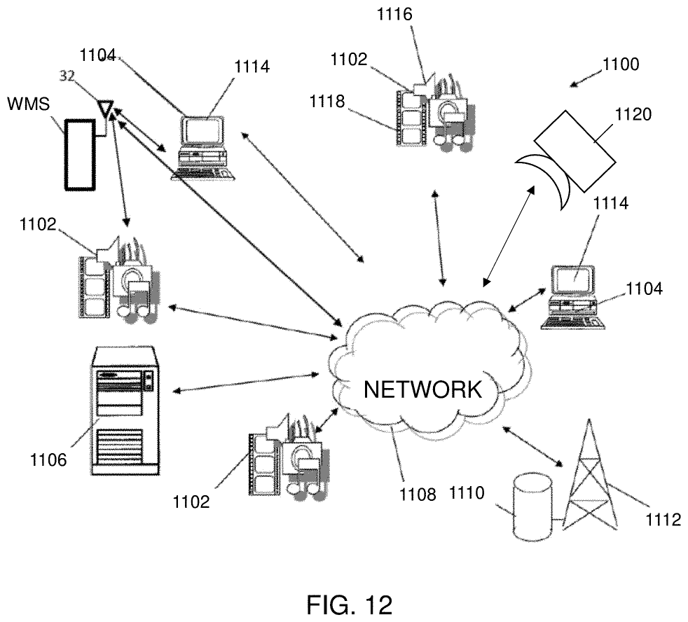

FIG. 12 is a schematic diagram illustrating an overall view of communication devices, computing devices, and mediums for implementing the wound monitoring system platform according to embodiments of the invention. The elements of the embodiments of FIGS. 7-11 are included in the networks and devices of FIG. 12.

The system 1100 includes multimedia devices 1102 and desktop computer devices 1104 configured with display capabilities 1114 and processors for executing instructions and commands. The multimedia devices 1102 are optionally mobile communication and entertainment devices, such as cellular phones and mobile computing devices that in certain embodiments are wirelessly connected to a network 1108. The multimedia devices 1102 typically have video displays 1118 and audio outputs 1116. The multimedia devices 1102 and desktop computer devices 1104 are optionally configured with internal storage, software, and a graphical user interface (GUI) for carrying out elements of the wound monitoring system platform according to embodiments of the invention. The network 1108 is optionally any type of known network including a fixed wire line network, cable and fiber optics, over the air broadcasts, satellite 1120, local area network (LAN), wide area network (WAN), global network (e.g., Internet), intranet, etc. with data/Internet and remote storage capabilities as represented by server 1106. Communication aspects of the network are represented by cellular base station 1110 and antenna 1112. In a preferred embodiment, the network 1108 is a LAN and each remote device 1102 and desktop device 1104 executes a user interface application (e.g., Web browser) to contact the server system 1106 through the network 1108. Alternatively, the remote devices 1102 and 1104 may be implemented using a device programmed primarily for accessing network 108 such as a remote client. Hermaticity/temperature/pressure/air quality sensors in the wound monitoring system may communicate directly or via the controller module M2 with remote devices 1102 and 1104 via near field communication standards such as Bluetooth or Zigbee, or alternatively via network 1108.

The software for the wound monitoring system platform, of certain inventive embodiments, is resident in the controller module M2, on multimedia devices 1102, desktop or laptop computers 1104, or stored within the server 1106 or cellular base station 1110 for download to an end user. Server 1106 may implement a cloud-based service for implementing embodiments of the platform with a multi-tenant database for storage of separate client data.

The present invention is further detailed with respect to the following non limiting examples. These examples are not intended to limit the scope of the invention but rather highlight properties of specific inventive embodiments and the superior performance thereof relative to comparative examples.

### EXAMPLES

### Example 1

Ten WMS systems were fabricated and tested for the ability to aspirate at three simulated exudate viscosities (100 cSt, 500 cSt, 1000 cSt). Algorithms based on relative humidity and temperature were tested to verify the ability to automatically control vacuum level. Algorithms based on air quality of the wound environment were tested to verify the ability to sniff the sampled air for chemicals associated with infection and then automatically introduce anti-infection treatments to the wound environment in response to detected infection indicating chemicals. The following dressing modules were tested: (1) controller simulator—electronics; (2) simulator—fluid reservoir to contain drainage solutions, vacuum port, humidity and temperature sensors, air quality sensor, pressure sensor port (for determining vacuum level), and flow restrictor port; (3) collection canister—hydrophobic filters for contamination control; and (4) heater module—to raise the exudate temperature to provide exudate evaporation and collection.

The ten systems were demonstrated to be capable of removing viscosities ranging from 100 cSt to 1000 cSt.

### Example 2

The ten WMS systems of example 1 demonstrated the ability to control humidity to 92%±2%, vacuum level to −125 mmHg±10 mmHg, temperatures of 99° F.±2° F., and air quality.

### Example 3

The ten WMS were evaluated to be capable of conforming to multiple anatomic locations and body types.

The dressing was shown to be fully compliant to conform to the contour of the PD catheter placement sites.

### Example 4

Stress tests were performed on the ten WMS and were run for 14-days (2-times the intended duration) to assure performance/reliability. Battery life was challenged under worst-case conditions of viscosity and vacuum leak. Software algorithms were developed for adaptive control and response to the delta pressure (P) and range over time relative to wound temperature and humidity.

### Example 5

A Design of Experiments (DOE) study was conducted to evaluate the ability to control pressure and the optimal conditions to remove the wound exudate, as well as, evaluate humidity sensor accuracy, evaluate wound temperature accuracy, evaluate air quality sensor accuracy, refine algorithms for balancing humidity, temperature, air quality, and vacuum level, evaluate fluid removal rate consistent with the wound types, and evaluate moisture levels and ability to dry with slow leak.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A wound management system for monitoring and controlling environmental conditions of a wound area of a patient comprising:
a wound dressing configured to be positioned over the wound area, said wound dressing defining a wound environment;
at least one of a humidity sensor, a pressure sensor, a temperature sensor, and a chemical sensor integrated into the wound dressing to provide physiologic parameters that correlate to a degree of wound healing;
a pump in fluid communication with said wound environment; and
a controller configured to control operation of said pump.

2. The system of claim 1 further comprising a strain relieving fixture.

3. The system of claim 1 further comprising an extrudate reservoir.

4. The system of claim 1 further comprising an air quality sensor integrated into the wound dressing to provide physiologic parameters indicative of infection.

5. The system of claim 1 wherein said wound dressing includes at least one port for introducing moisture or an anti-infection treatment into the wound environment.

6. The system of claim 1 wherein said wound dressing accommodates a variation in catheter diameters.

7. The system of claim 1 wherein said at least one humidity sensor, pressure sensor, or temperature sensor determine a degree of wound hermaticity via measurements of humidity in a vacuum line.

8. The system of claim 1 wherein said at least one humidity sensor, pressure sensor, or temperature sensor determine a degree of wound hermaticity via measurements of local tissue oxygenation in the immediate vicinity of said wound in a patient's skin.

9. The system of claim 1 wherein said environmental conditions are communicated by wired or wireless connection to a computing or a communication device for immediate or remote monitoring.

10. The system of claim 1 wherein said at least one sensors require an external power source and said external power source is a battery used to supply a vacuum source.

11. The system of claim 1 wherein said at least one sensors are passive elements which do not require an external power source.

12. The system of claim 1 wherein said physiologic parameters are employed for local closed-loop control of a vacuum supply to said wound dressing.

13. The system of claim 1 further comprising an observation window in said dressing providing a view of the wound.

14. The system of claim 1 wherein the controller has a microprocessor that receives input from said at least one sensors and compares the signals to a set of controller sensors to adjust the vacuum levels.

15. The system of claim 1 wherein the controller controls a diaphragm pump/motor assembly, a display screen, a pressure sensor, and a slow-leak flow restrictor.

16. A method for measuring and monitoring wound conditions of a patient comprising:
placing one or more sensors for measuring parameters that correlate to a degree of wound healing at a wound area of a patient; and
wherein said one or more sensors are incorporated into a wound dressing positioned to cover the wound area.

17. The method of claim 16 wherein said one or more sensors determine said degree of wound healing via measurements of humidity, temperature, air quality, and pressure in a vacuum line to said wound dressing.

18. The method of claim 16 wherein said one or more sensors determine a degree of wound hermaticity via measurements of local tissue oxygenation in the immediate vicinity of the wound area on the patient's skin.

19. The method of claim 16 wherein said wound conditions are communicated by wired or wireless connection to a controller, computing, or a communication device for immediate or remote monitoring.

20. The method of claim 16 wherein said sensor parameters are employed for local closed-loop control of a vacuum supply to said wound dressing.
